(19) United States
(12) Patent Application Publication
(10) Pub. No.: US 2016/0370571 A1
(43) Pub. Date: Dec. 22, 2016

(54) STEREOSCOPIC IMAGING OPTICAL SYSTEM, STEREOSCOPIC IMAGING DEVICE, AND ENDOSCOPE

(71) Applicant: OLYMPUS CORPORATION, Tokyo (JP)

(72) Inventor: Takayoshi TOGINO, Tokyo (JP)

(73) Assignee: OLYMPUS CORPORATION, Tokyo (JP)

(21) Appl. No.: 15/250,344

(22) Filed: Aug. 29, 2016

Related U.S. Application Data
(63) Continuation of application No. PCT/JP2015/052263, filed on Jan. 28, 2015.

(30) Foreign Application Priority Data
Apr. 24, 2014 (JP) 2014-089763

(51) Int. Cl. G02B 23/24; G02B 27/00; G02B 5/04; A61B 1/00; H04N 13/02

(52) U.S. Cl. CPC G02B 23/2415; A61B 1/00193; H04N 13/0217; G02B 5/04; G02B 27/0025

(57) ABSTRACT

A first central principal ray Lc1 of a first light beam L1 that has passed through a first front group Gf1 passes through a back first group Gb1, a first aperture center CS1, a first deflection group Gv1, and a back second group Gb2 at a position separated from a back group central axis Cb and reaches a image plane I, and a second central principal ray Lc2 of a second light beam L2 that has passed through a second front group Gf2 passes through the back first group Gb1, a second aperture center CS2, a second deflection group Gv2, and the back second group Gb2 at a position separated from the back group central axis Cb and reaches the image plane I.

### CROSS-REFERENCE TO RELATED APPLICATIONS

This application is a continuation claiming priority on the basis of Japan Patent Application No. 2014-089763 applied in Japan on Apr. 24, 2014 and based on PCT/JP2015/052263 filed on Jan. 28, 2015. The contents of both the PCT application and the Japan Application are incorporated herein by reference.

### BACKGROUND OF THE INVENTION AND RELATED ART STATEMENT

The present invention relates to a stereoscopic imaging optical system, a stereoscopic imaging device, and an endoscope.

There is conventionally disclosed a method of forming a stereoscopic image using two images having different parallaxes made to be formed on substantially the same plane for imaging (see JP 08-122665A, Japanese Patent No. 4,248,771, Japanese Patent No. 4,093,503 and JP 2001-147382A).

### SUMMARY OF INVENTION

A stereoscopic imaging optical system according to an embodiment of the present invention includes in order from an object side to an image plane side: a front group having a first front group centered about a first front group central axis and a second front group centered about a second front group central axis extending parallel to the first front group central axis; and a back group centered about a single back group central axis extending parallel to the first front group central axis and second front group central axis. The back group includes: a back first group on the object side; a back second group on the image side; a first aperture disposed between the back first group and back second group and centered about a first aperture center offset from the back group central axis; a second aperture centered about a second aperture center disposed at a position plane-symmetric to the first aperture center with respect to a plane perpendicular to a plane including the first front group central axis and second front group central axis and including the back group central axis; a first deflection group disposed between the back first group and back second group; and a second deflection group disposed at a position plane-symmetric to the first deflection group with respect to a plane perpendicular to a plane including the first front group central axis and second front group central axis and including the back group central axis. The first central principal ray of a first light beam that has passed through the first front group passes through the back first group, first aperture center, first deflection group, and the back second group at a position separated from the back group central axis and reaches the image plane, and a second central principal ray of a second light beam that has passed through the second front group passes through the back first group, second aperture center, second deflection group, and the back second group at a position separated from the back group central axis and reaches the image plane.

A stereoscopic imaging device according to an embodiment of the present invention includes the above stereoscopic imaging optical system and an imaging device.

An endoscope according to an embodiment of the present invention includes the above stereoscopic imaging device.

### BRIEF DESCRIPTION OF DRAWINGS

FIG. 1 is a cross-sectional view of a stereoscopic imaging optical system according to an embodiment taken along a central axis thereof;

FIG. 2 is a view illustrating an example in which a deflection group of the stereoscopic imaging optical system according to the embodiment is formed into a wedge prism shape;

FIG. 3 is a view illustrating another example in which a deflection group of the stereoscopic imaging optical system according to the embodiment is formed into a wedge prism shape;

FIG. 4 is a cross-sectional view of a stereoscopic imaging optical system of Example 1 taken along a plane including a first front group central axis and a second front group central axis;

FIG. 5 is a cross-sectional view of the stereoscopic imaging optical system of Example 1 taken along a plane perpendicular to a plane including the first front group central axis and second front group central axis and including a back group central axis;

FIG. 6 is a lateral aberration diagram of the stereoscopic imaging optical system of Example 1;

FIG. 7 is a lateral aberration diagram of the stereoscopic imaging optical system of Example 1;

FIG. 8 is a cross-sectional view of a stereoscopic imaging optical system of Example 2 taken along a plane including the first front group central axis and second front group central axis;

FIG. 9 is a cross-sectional view of the stereoscopic imaging optical system of Example 2 taken along a plane perpendicular to a plane including the first front group central axis and second front group central axis and including the back group central axis;

FIG. 10 is a lateral aberration diagram of the stereoscopic imaging optical system of Example 2;

FIG. 11 is a lateral aberration diagram of the stereoscopic imaging optical system of Example 2;

FIG. 12 is a cross-sectional view of a stereoscopic imaging optical system of Example 3 taken along a plane including the first front group central axis and second front group central axis;

FIG. 13 is a cross-sectional view of the stereoscopic imaging optical system of Example 3 taken along a plane perpendicular to a plane including the first front group central axis and second front group central axis and including the back group central axis;

FIG. 14 is a lateral aberration diagram of the stereoscopic imaging optical system of Example 3;

FIG. 15 is a lateral aberration diagram of the stereoscopic imaging optical system of Example 3;

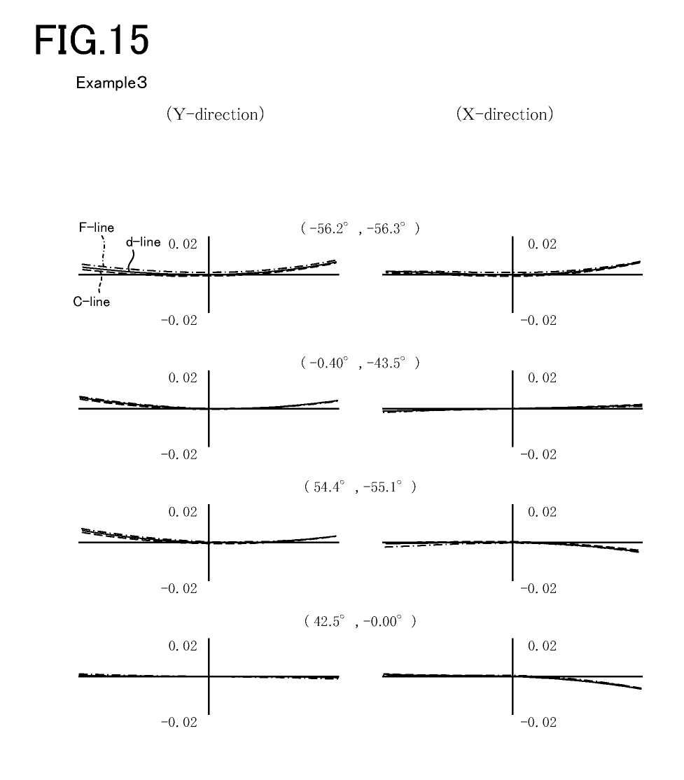

FIG. 16 is a view schematically illustrating an example in which the stereoscopic imaging optical system of the present embodiment is used for a stereoscopic imaging device;

FIGS. 17A and 17B are views illustrating an example in which the stereoscopic imaging optical system according to the present embodiment is attached to a distal end of an endoscope; and

FIG. 18 is an example in which the stereoscopic imaging optical system according to the present embodiment is attached to a distal end of a flexible electronic endoscope.

### DESCRIPTION OF EMBODIMENTS

Hereinafter, a stereoscopic imaging optical system 1 according to an embodiment will be described.

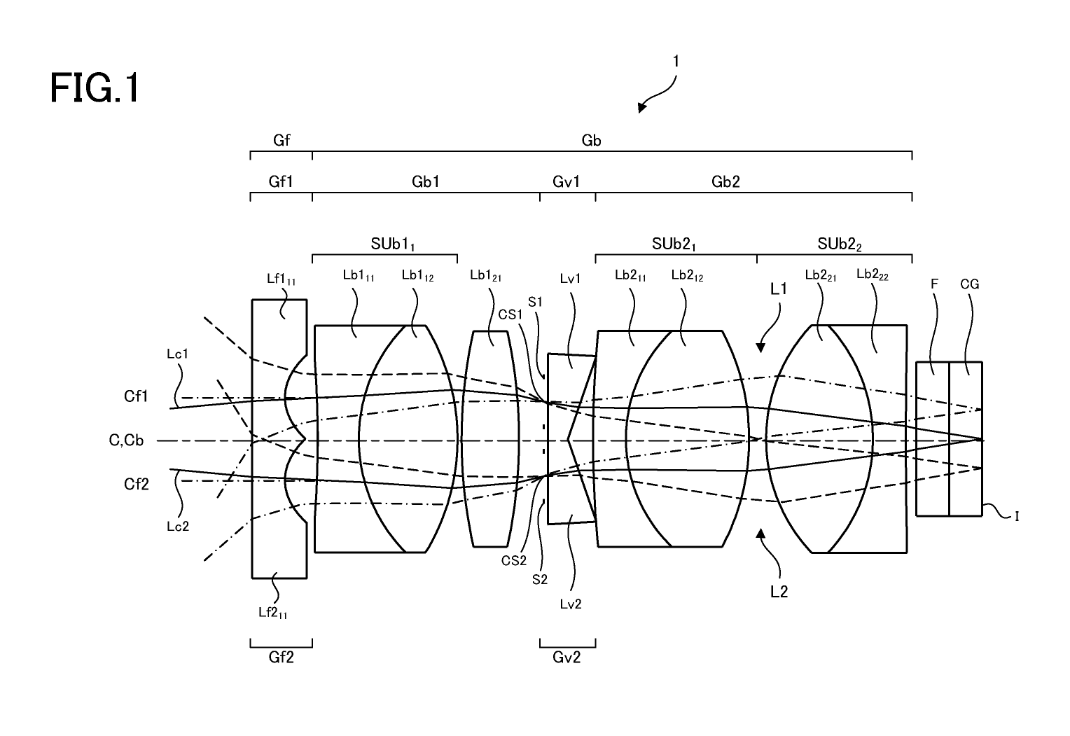

FIG. 1 is a cross-sectional view of the stereoscopic imaging optical system 1 according to an embodiment taken along a central axis C thereof.

The stereoscopic imaging optical system 1 according to the present embodiment includes a front group Gf and a back group Gb, in order from an object side to an image plane I side. The front group Gf includes a first front group Gf1 centered about a first front group central axis Cf1 and a second front group Gf2 centered about a second front group central axis Cf2 extending parallel to the first front group central axis Cf1. The back group Gb is centered about a single back group central axis Cb extending parallel to the first front group central axis Cf1 and second front group central axis Cf2. The back group Gb includes a back first group Gb1 on the object side, a back second group Gb2 on the image side, a first aperture S1 disposed between the back first group Gb1 and back second group Gb2 and centered about a first aperture center CS1 offset from the back group central axis Cb, a second aperture S2 centered about a second aperture center CS2 disposed at a position plane-symmetric to the first aperture center CS1 with respect to a plane perpendicular to a plane including the first front group central axis Cf1 and second front group central axis Cf2 and including the back group central axis Cb, a first deflection group Gv1 disposed between the back first group Gb1 and back second group Gb2, and a second deflection group Gv2 disposed at a position plane-symmetric to the first deflection group Gv1 with respect to a plane perpendicular to a plane including the first front group central axis Cf1 and second front group central axis Cf2 and including the back group central axis Cb. A first central principal ray Lc1 of a first light beam L1 that has passed through the first front group Gf1 passes through the back first group Gb1, first aperture center CS1, first deflection group Gv1, and the back second group Gb2 at a position separated from the back group central axis Cb and reaches the image plane I. A second central principal ray Lc2 of a second light beam L2 that has passed through the second front group Gf2 passes through the back first group Gb1, second aperture center CS2, second deflection group Gv2, and back second group Gb2 at a position separated from the back group central axis Cb and reaches an image plane I.

The first aperture center CS1 may be included in an extension of the first front group central axis Cf1, and the second aperture center CS2 may be included in an extension of the second front group central axis Cf2.

In the stereoscopic imaging optical system 1 according to the present embodiment, the back first group Gb1 and back second group Gb2 are formed in a rotational symmetry with respect to the single back group central axis Cb, so that the first front group central axis Cf1 and second front group central axis Cf2 can be brought close to each other. Further, the first central principal ray Lc1 of the first light beam L1 that has passed through the first front group Gf1 passes through the back first group Gb1, first aperture center CS1, first deflection group Gv1, and the back second group Gb2 at a position separated from the back group central axis Cb and reaches the image plane I, and second central principal ray Lc2 of the second light beam L2 that has passed through the second front group Gf2 passes through the back first group Gb1, second aperture center CS2, second deflection group Gv2, and the back second group Gb2 at a position separated from the back group central axis Cb and reaches the image plane I, so that aberration generated upon passage through the back first group Gb1 can be corrected in the back second group Gb2.

Further, in the stereoscopic imaging optical system 1 according to the present embodiment, the first aperture S1 and first deflection group Gv1 are disposed adjacent to each other, and the second aperture S2 and second deflection group Gv2 are disposed adjacent to each other.

Portions around the first aperture S1 and second aperture S2 are portions at which the first light beam L1 and second light beam L2 are collected, respectively, and effective diameters of the light beams L1 and L2 become smallest. This allows effective diameters of the first deflection group Gv1 and second deflection group Gv2 to be reduced. This can further reduce a distance between the first aperture S1 and second aperture S2 disposed in parallel, making the back first group Gb1 and back second group Gb2 small, which allows a reduction in the size of the entire back group Gb.

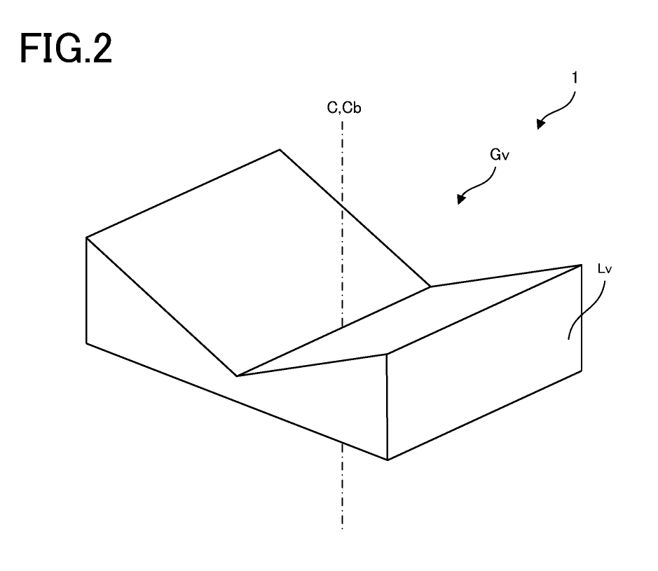

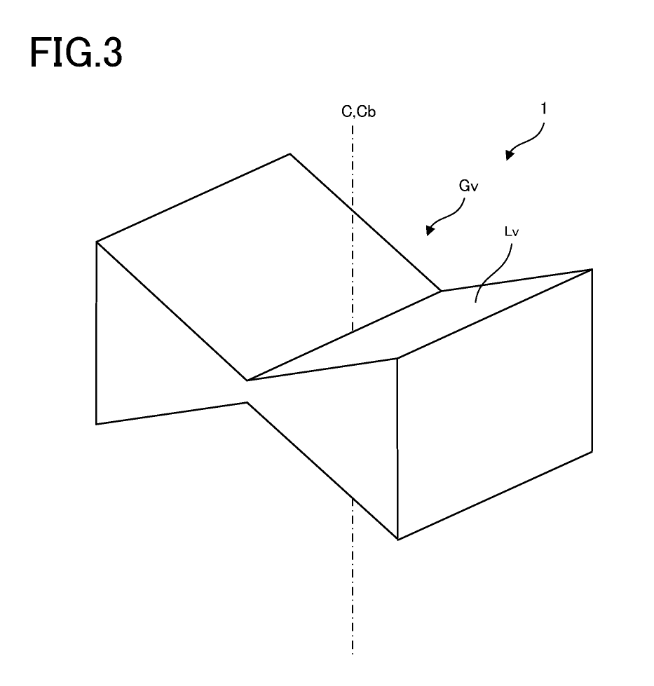

FIG. 2 is a view illustrating an example in which the back deflection group Gv of the stereoscopic imaging optical system 1 according to the embodiment is formed into a wedge prism shape. FIG. 3 is a view illustrating another example in which the back deflection group Gv of the stereoscopic imaging optical system 1 according to the embodiment is formed into a wedge prism shape.

In the stereoscopic imaging optical system 1 according to the present embodiment, the first deflection group Gv1 and second deflection group Gv2 are constituted of optical elements Lv1 and Lv2, respectively, whose thickness in the back group central axis Cb direction gradually increases in a direction separating from the back group central axis Cb.

The first light beam L1 and second light beam L2 can be made to pass near the back group central axis Cb in the back second group Gb2, thereby allowing aberration correction ability of the back second group Gb2 to be enhanced. The optical elements Lv1 and Lv2 may be separately formed as a first optical element Lv1 and a second optical element Lv2.

Further, in the stereoscopic imaging optical system 1 according to the present embodiment, the optical elements Lv1 and Lv2 have a wedge prism shape.

Forming the optical elements Lv1 and Lv2 into a wedge prism shape allows both surfaces of each of the optical elements Lv1 and Lv2 to be formed as a plane, thereby making it possible to improve workability.

Further, in the stereoscopic imaging optical system 1 according to the present embodiment, the first front group Gf1 and second front group Gf2 are constituted of parallel-arranged concave lenses having the same shape.

Therefore, it is possible to suppress different image distortions from occurring in light paths of the respective first and second light beams L1 and L2. Further, the first front group Gf1 and second front group Gf2 each have a lens whose object side surface has a plane or a convex surface facing the object side and whose image plane side has a strong concave surface, thereby making it possible to reduce occurrence of a rotationally asymmetric image distortion.

Further, in the stereoscopic imaging optical system 1 according to the present embodiment, the parallel-arranged concave lenses are formed integrally.

Integrally forming the front group Gf can reduce an optical-axis interval, thereby further reducing the size of the stereoscopic imaging optical system 1.

Further, the stereoscopic imaging optical system 1 according to the present embodiment satisfies the following conditional formula (1):

3<fl/d<5 (1)

where fl is the entire length of the optical system, and d is the maximum outer diameter of the optical system.

When the lower limit of the conditional formula (1) is exceeded, the maximum outer diameter of the stereoscopic imaging optical system 1 is increased to disadvantageously enlarge the stereoscopic imaging optical system 1. When the upper limit of the conditional formula (1) is exceeded, the entire length of the stereoscopic imaging optical system 1 is increased to disadvantageously enlarge the stereoscopic imaging optical system 1.

Further, in the stereoscopic imaging optical system 1 according to the present embodiment, an interval between the first front group central axis Cf1 and second front group central axis Cf2 is set to equal to or less than 1.2 mm.

In general, the shortest distance at which we can see things stereoscopically is about 30 cm. At a distance shorter than this, it is difficult to adjust the eyes, resulting in the condition of out-of-focus. Assuming that an eye width is 6 cm, a convergence angle becomes 6°. When stereoscopic viewing is performed with a convergence angle of 6° or greater, we feel as if we saw a miniature or we ourselves became a giant due to homeostasis in terms of size.

When we observe an object in an enlarged manner by approaching the object as in the present embodiment, the optical axis interval between both eyes and an object distance are determined by the convergence angle. For example, when the object distance is 10 mm, 2 mm is required for the optical axis interval; when the object distance is 6 mm, 1.2 mm is required for the optical axis interval. That is, for performing enlarging observation with an object distance of 6 mm, the optical axis interval needs to be 1.2 mm, at which the convergence angle becomes 6° or smaller.

Hereinafter, Examples 1 to 3 of the stereoscopic imaging optical system 1 according to the present embodiment will be described. Numerical data of the Examples 1 to 3 will be given later.

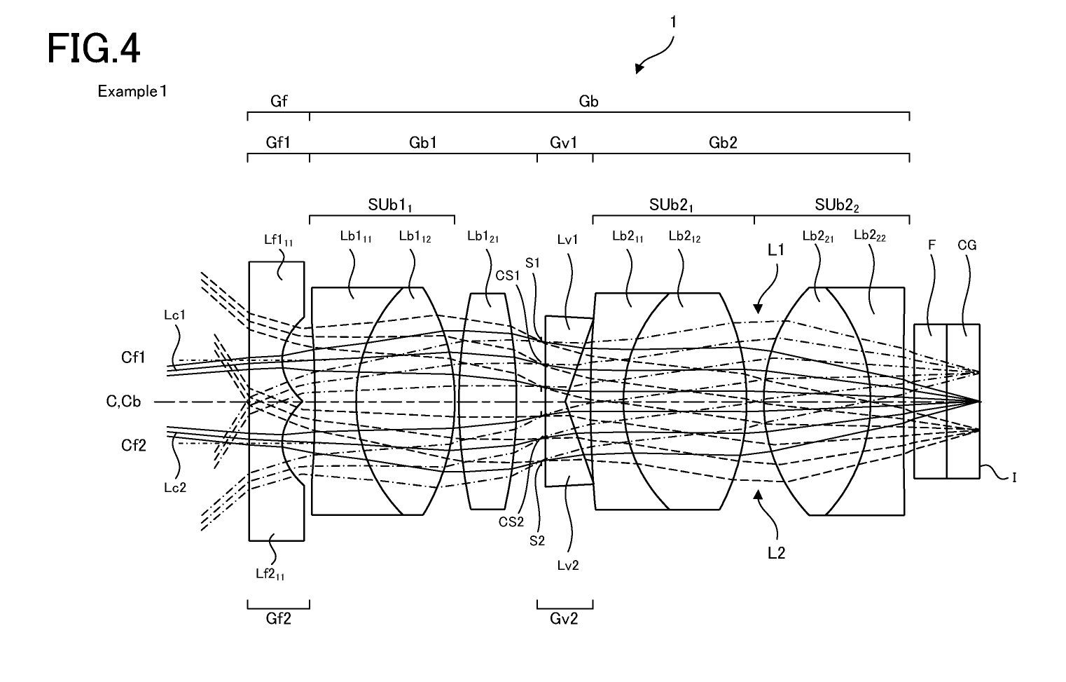

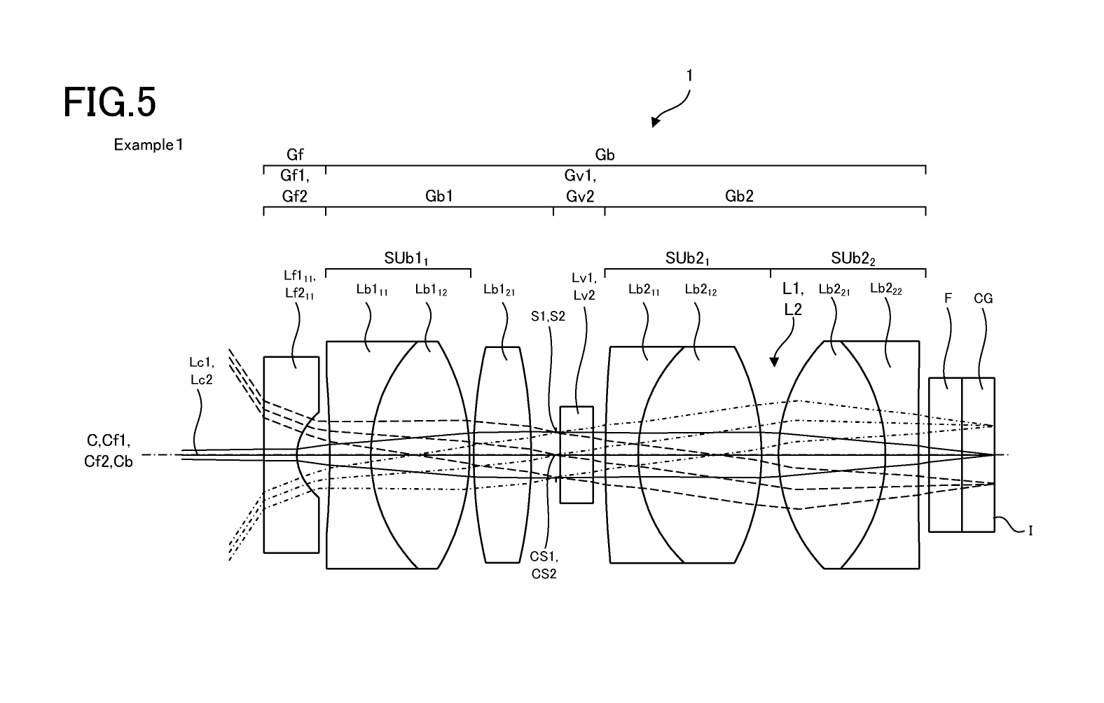

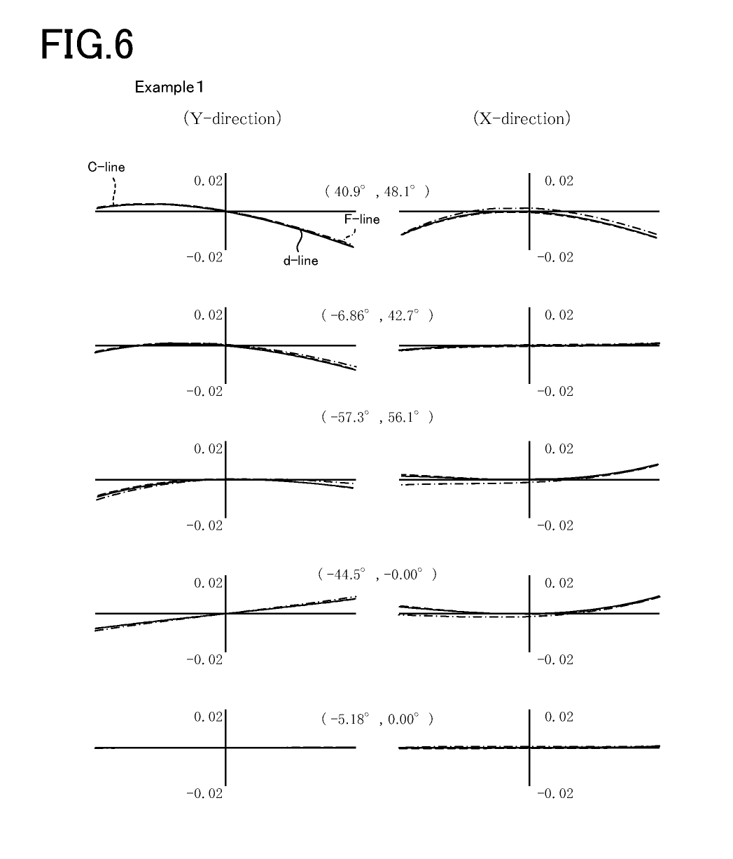

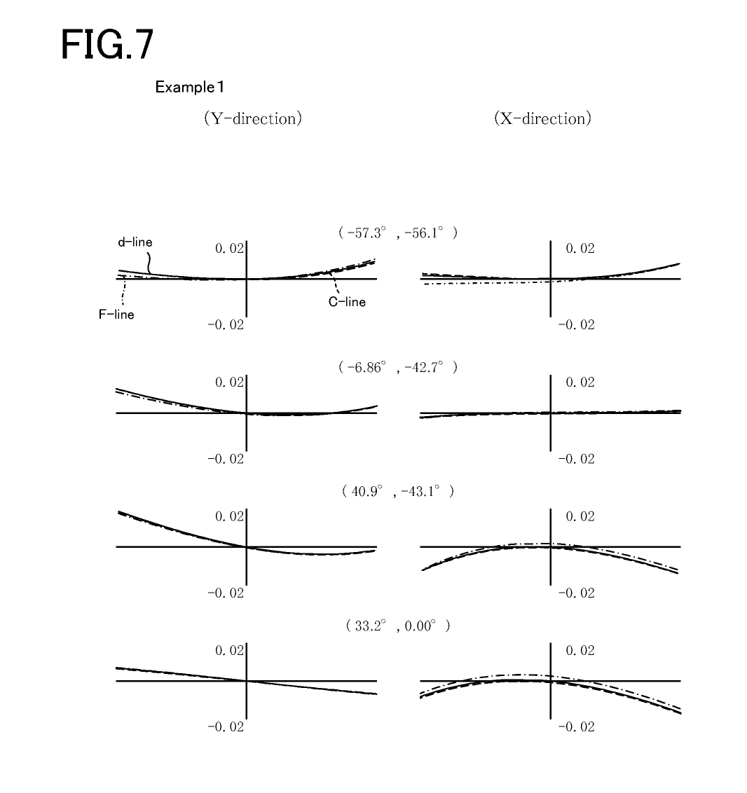

FIG. 4 is a cross-sectional view of the stereoscopic imaging optical system 1 of Example 1 taken along a plane including the first front group central axis Cf1 and second front group central axis Cf2. FIG. 5 is a cross-sectional view of the stereoscopic imaging optical system 1 of Example 1 taken along a plane perpendicular to a plane including the first front group central axis Cf1 and second front group central axis Cf2 and including the back group central axis Cb. FIG. 6 is a lateral aberration diagram of the stereoscopic imaging optical system 1 of Example 1. FIG. 7 is a lateral aberration diagram of the stereoscopic imaging optical system 1 of Example 1.

In the lateral aberration diagram, angles shown in a center of the drawing indicate (view angles in a vertical direction), and lateral aberrations at the angles in a Y-direction (meridional direction) and an X-direction (sagittal direction) are illustrated. A negative view angle means a clockwise angle with respect to an X-axis positive direction. The same applies to the lateral aberration diagrams of Examples 1 to 3.

As illustrated in FIG. 4, the stereoscopic imaging optical system 1 according to Example 1 includes a front group Gf and a back group Gb, in order from an object side to an image side. The front group Gf includes a first front group Gf1 having a first front group central axis Cf1 and a second front group Gf2 having a second front group central axis Cf2 extending parallel to the first front group central axis Cf1. The back group Gb has a single back group central axis Cb.

Parallel arrangement of the first front group Gf1 and second front group Gf2 allows stereoscopic observation.

The first front group Gf1 has a flat-concave negative lens Lf1₁₁ whose flat surface faces the object side. The second front group Gf2 has a flat-concave negative lens Lf2₁₁ whose flat surface faces the object side. The first front group Gf1 and second front group Gf2 are preferably formed integrally into the same shape.

The back group Gb includes a back first group Gb1, a back second group Gb2, a first aperture S1, a second aperture S2, a first deflection group Gv1, and a second deflection group Gv2. The back first group Gb1 has: a cemented lens SUb1₁ composed of a concave-concave negative lens Lb1₁₁ and a convex-convex positive lens Lb1₁₂; and a convex-convex positive lens Lb1₂₁. The back second group Gb2 has: a cemented lens SUb2₁ composed of a negative meniscus lens Lb2₁₁ whose convex surface faces the object side and a convex-convex positive lens Lb2₁₂; and a cemented lens SUb2₂ composed of a convex-convex positive lens Lb2₂₁ and a concave-concave negative lens Lb2₂₂. The first aperture S1 is disposed between the back first group Gb1 and back second group Gb2 and centered about a first aperture center CS1 offset from the back group central axis Cb. The second aperture S2 is centered about a second aperture center CS2 disposed at a position plane-symmetric to the first aperture center CS1 with respect to a plane perpendicular to a plane including the first front group central axis Cf1 and second front group central axis Cf2 and including the back group central axis Cb. The first deflection group Gv1 is disposed between the back first group Gb1 and back second group Gb2. The second deflection group Gv2 is disposed at a position plane-symmetric to the first deflection group Gv1 with respect to a plane perpendicular to a plane including the first front group central axis Cf1 and second front group central axis Cf2 and including the back group central axis Cb.

The first aperture S1 and first deflection group Gv1 are disposed adjacent to each other, and the second aperture S2 and second deflection group Gv2 are disposed adjacent to each other. In Example 1, the first aperture S1 is disposed on the object side of the first deflection group Gv1, and the second aperture S2 is disposed on the object side of the second deflection group Gv2.

The first and second deflection groups Gv1 and Gv2 of Example 1 are each constituted of a wedge prism shaped optical element whose thickness in the back group central axis Cb direction gradually increases in a direction separating from the back group central axis Cb. Further, the wedge prism shaped optical elements constituting the first and second deflection groups Gv1 and Gv2 of Example 1 are integrally formed. The integrally formed optical element of Example 1 has an object side surface formed into a plane perpendicular to the back group central axis Cb and an image plane side surface formed into a plane inclined relative to the back group central axis Cb.

Further, a filter F and a cover glass CG are disposed immediately in front of the image plane I.

A first light beam L1 that has entered the first front group Gf1 of the front group Gf from an unillustrated first object plane passes through the flat-concave negative lens Lf1₁₁ to exit from the first front group Gf1 and then enters the back group Gb. The first light beam L1 that has entered the back first group Gb1 of the back group Gb passes through the cemented lens SUb1₁ and convex-convex positive lens Lb1₂₁ to exit from the back first group Gb1 and then passes through the first aperture S1. The first light beam L1 that has passed through the first aperture S1 passes through the first deflection group Gv1 and enters the back second group Gb2. The first light beam L1 that has entered the back second group Gb2 passes through the cemented lens SUb2₁ and cemented lens SUb2₂ to exit from the back second group Gb2, passes through the filter F and cover glass CG, and reaches the image plane I.

A second light beam L2 that has entered the second front group Gf2 of the front group Gf from an unillustrated second object plane passes through the flat-concave negative lens Lf2₁₁ to exit from the second front group Gf2 and then enters the back group Gb. The second light beam L2 that has entered the back first group Gb1 of the back group Gb passes through the cemented lens SUb1₁ and convex-convex positive lens Lb1₂₁ to exit from the back first group Gb1 and then passes through the second aperture S2. The second light beam L2 that has passed through the second aperture S2 passes through the second deflection group Gv2 and enters the back second group Gb2. The second light beam L2 that has entered the back second group Gb2 passes through the cemented lens SUb2₁ and cemented lens SUb2₂ to exit from the back second group Gb2, passes through the filter F and cover glass CG, and reaches the image plane I.

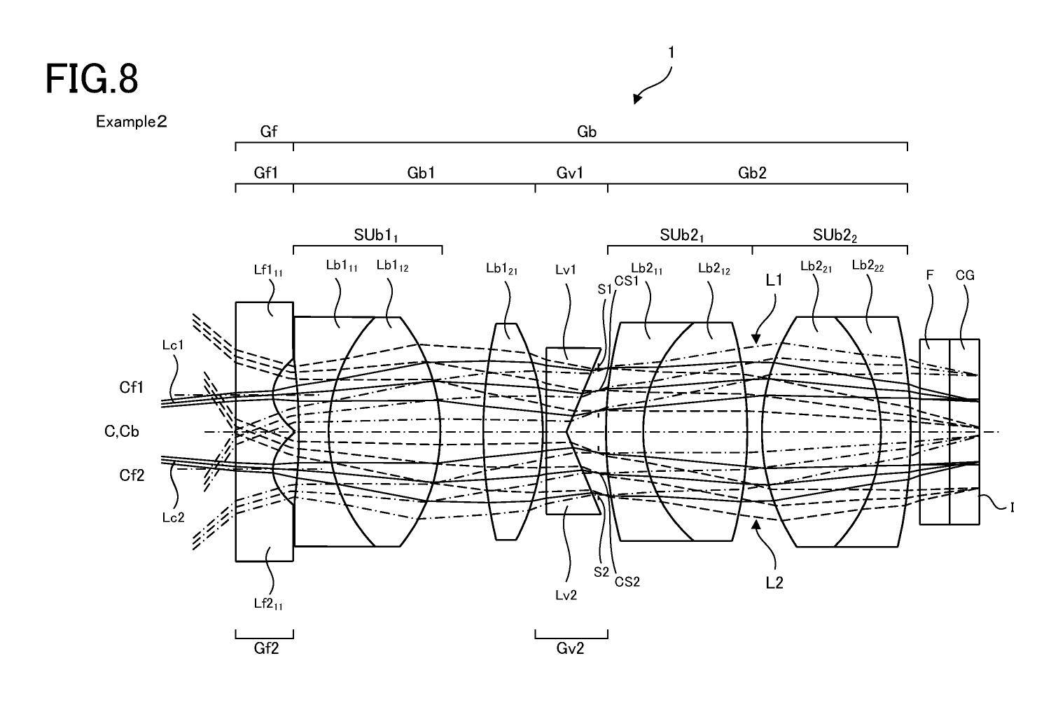

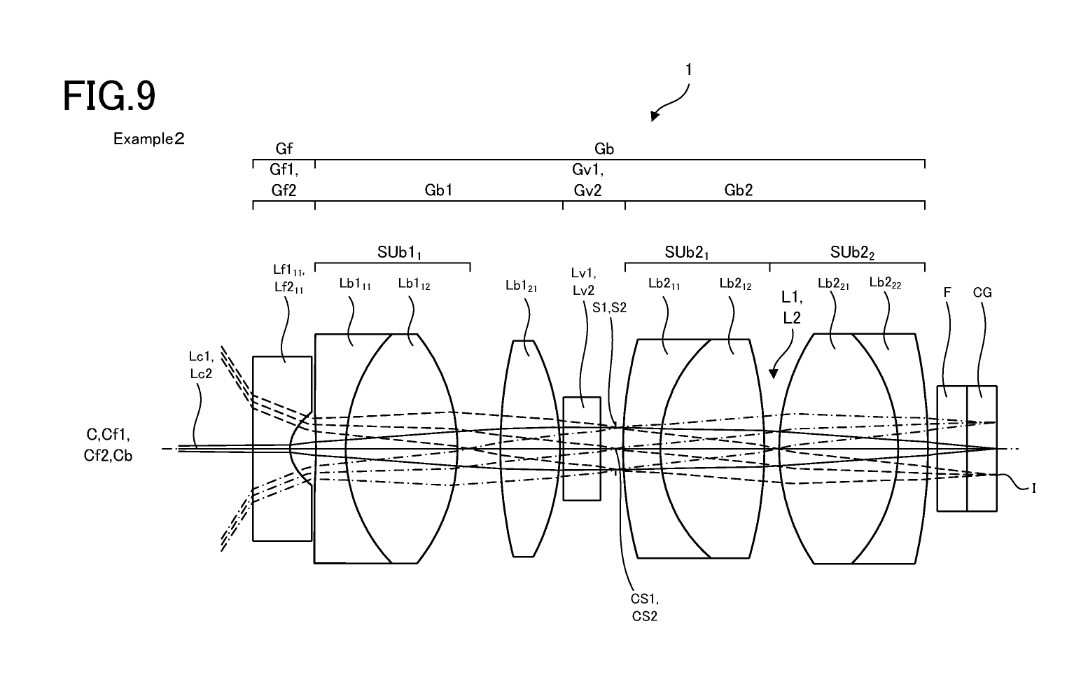

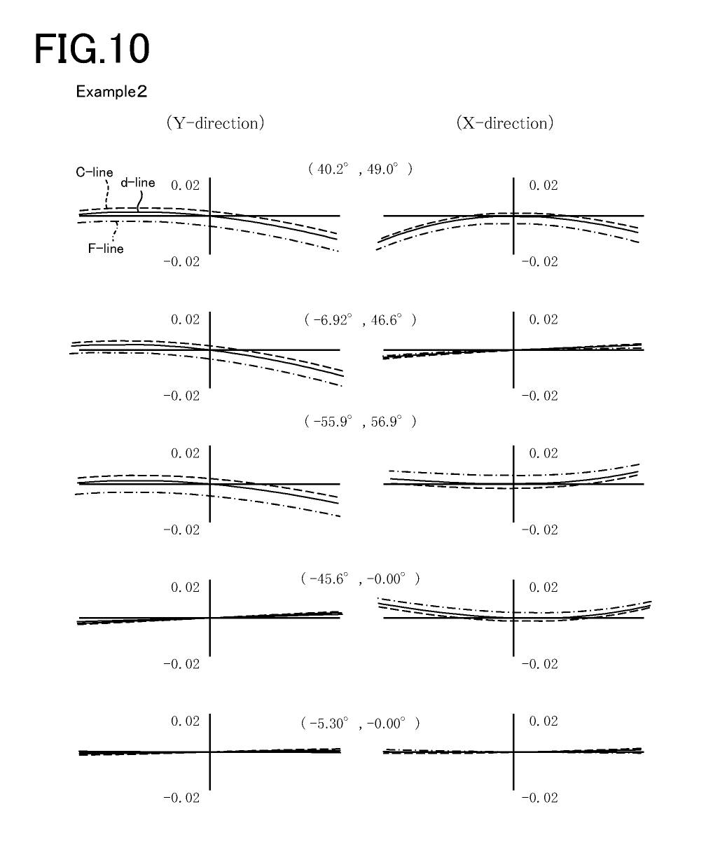

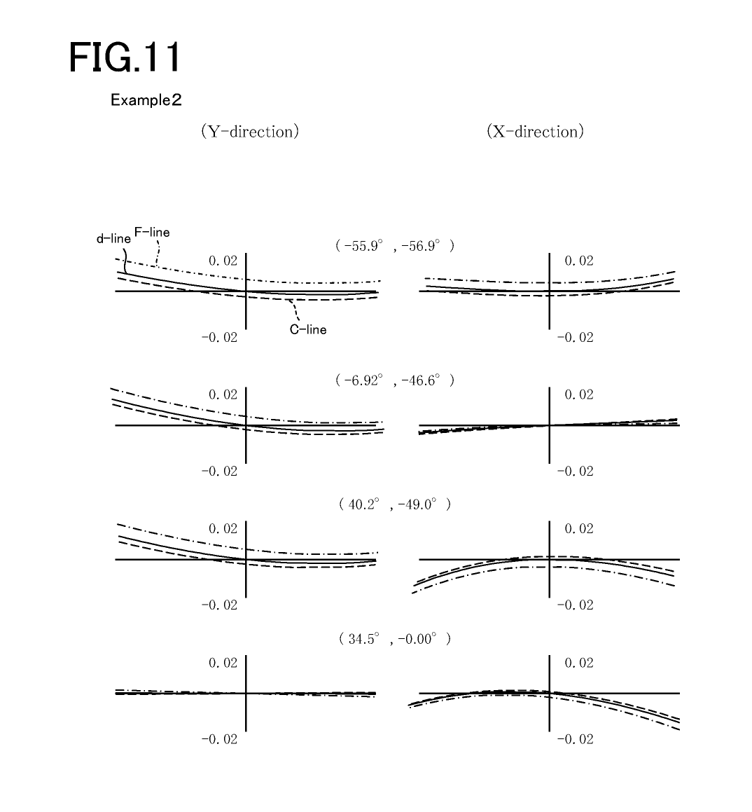

FIG. 8 is a cross-sectional view of the stereoscopic imaging optical system 1 of Example 2 taken along a plane including the first front group central axis Cf1 and second front group central axis Cf2. FIG. 9 is a cross-sectional view of the stereoscopic imaging optical system 1 of Example 2 taken along a plane perpendicular to a plane including the first front group central axis Cf1 and second front group central axis Cf2 and including the back group central axis Cb. FIG. 10 is a lateral aberration diagram of the stereoscopic imaging optical system 1 of Example 2. FIG. 11 is a lateral aberration diagram of the stereoscopic imaging optical system 1 of Example 2.

As illustrated in FIG. 8, the stereoscopic imaging optical system 1 according to Example 2 includes a front group Gf and a back group Gb, in order from an object side ton an image side. The front group Gf includes a first front group Gf1 having a first front group central axis Cf1 and a second front group Gf2 having a second front group central axis Cf2 extending parallel to the first front group central axis Cf1. The back group Gb has a single back group central axis Cb.

Parallel arrangement of the first front group Gf1 and second front group Gf2 allows stereoscopic observation.

The first front group Gf1 has a flat-concave negative lens Lf1₁₁ whose flat surface faces the object side. The second front group Gf2 has a flat-concave negative lens Lf2₁₁ whose flat surface faces the object side. The first front group Gf1 and second front group Gf2 are preferably formed integrally into the same shape.

The back group Gb includes a back first group Gb1, a back second group Gb2, a first deflection group Gv1, a second deflection group Gv2, a first aperture S1, and a second aperture S2. The back first group Gb1 has: a cemented lens SUb1₁ composed of a concave-concave negative lens Lb1₁₁ and a convex-convex positive lens Lb1₁₂; and a convex-convex positive lens Lb1₂₁. The back second group Gb2 has: a cemented lens SUb2₁ composed of a negative meniscus lens Lb2₁₁ whose convex surface faces the object side and a convex-convex positive lens Lb2₁₂; and a cemented lens SUb2₂ composed of a convex-convex positive lens Lb2₂₁ and a negative meniscus lens Lb2₂₂ whose convex surface faces the image plane I side. The first deflection group Gv1 is disposed between the back first group Gb1 and back second group Gb2. The second deflection group Gv2 is disposed at a position plane-symmetric to the first deflection group Gv1 with respect to a plane perpendicular to a plane including the first front group central axis Cf1 and second front group central axis Cf2 and including the back group central axis Cb. The first aperture S1 is disposed between the back first group Gb1 and back second group Gb2 and centered about a first aperture center CS1 offset from the back group central axis Cb. The second aperture S2 is centered about a second aperture center CS2 disposed at a position plane-symmetric to the first aperture center CS1 with respect to a plane perpendicular to a plane including the first front group central axis Cf1 and second front group central axis Cf2 and including the back group central axis Cb.

The first aperture S1 and first deflection group Gv1 are disposed adjacent to each other, and second aperture S2 and second deflection group Gv2 are disposed adjacent to each other. In Example 2, the first aperture S1 is disposed on the image side of the first deflection group Gv1, and second aperture S2 is disposed on the image side of the second deflection group Gv2.

The first and second deflection groups Gv1 and Gv2 of Example 2 are each constituted of a wedge prism shaped optical element whose thickness in the back group central axis Cb direction gradually increases in a direction separating from the back group central axis Cb. Further, the wedge prism shaped optical elements constituting the first and second deflection groups Gv1 and Gv2 of Example 2 are integrally formed. The integrally formed optical element of Example 2 has an object side surface formed into a plane perpendicular to the back group central axis Cb and an image plane side surface formed into a plane inclined relative to the back group central axis Cb.

Further, a filter F and a cover glass CG are disposed immediately in front of the image plane I.

A first light beam L1 that has entered the first front group Gf1 of the front group Gf from an unillustrated first object plane passes through the flat-concave negative lens Lf1₁₁ to exit from the first front group Gf1 and then enters the back group Gb. The first light beam L1 that has entered the back first group Gb1 of the back group Gb passes through the cemented lens SUb1₁ and convex-convex positive lens Lb1₂₁ to exit from the back first group Gb1 and then passes through the first deflection group Gv1. The first light beam L1 that has passed through the first deflection group Gv1 passes through the first aperture S1 and enters the back second group Gb2. The first light beam L1 that has entered the back second group Gb2 passes through the cemented lens SUb2₁ and cemented lens SUb2₂ to exit from the back second group Gb2, passes through the filter F and cover glass CG, and reaches the image plane I.

A second light beam L2 that has entered the second front group Gf2 of the front group Gf from an unillustrated second object plane passes through the flat-concave negative lens Lf2₁₁ to exit from the second front group Gf2 and then enters the back group Gb. The second light beam L2 that has entered the back first group Gb1 of the back group Gb passes through the cemented lens SUb1₁ and convex-convex positive lens Lb1₂₁ to exit from the back first group Gb1 and then passes through the second deflection group Gv2. The second light beam. L2 that has passed through the second deflection group Gv2 passes through the second aperture S2 and enters the back second group Gb2. The second light beam L2 that has entered the back second group Gb2 passes through the cemented lens SUb2₁ and cemented lens SUb2₂ to exit from the back second group Gb2, passes through the filter F and cover glass CG, and reaches the image plane I.

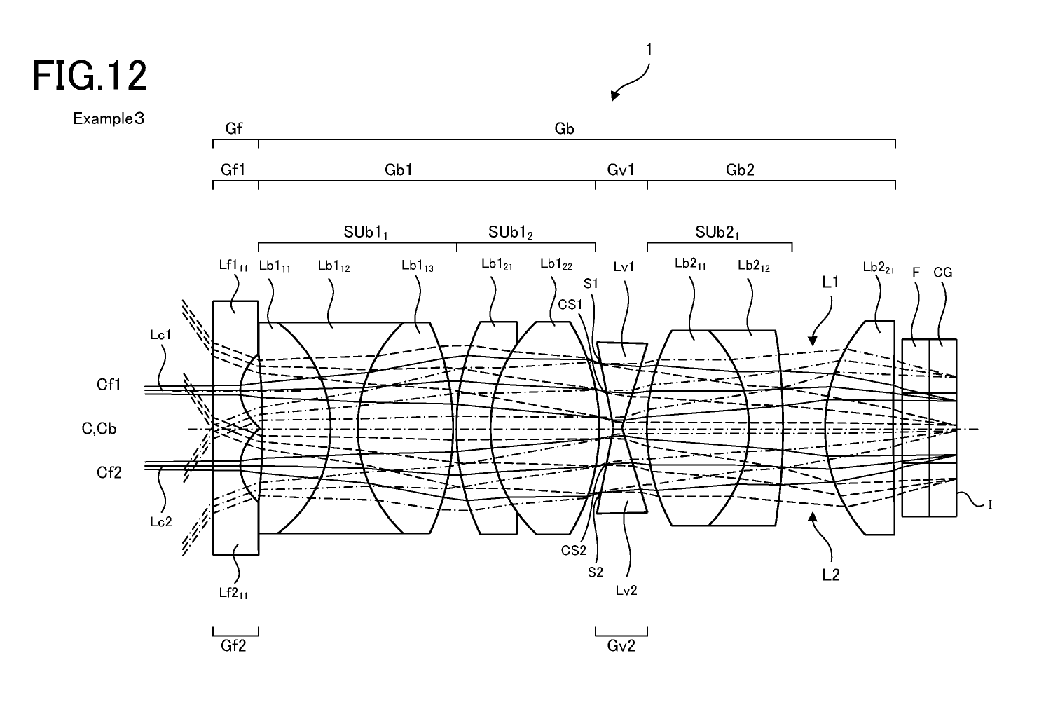

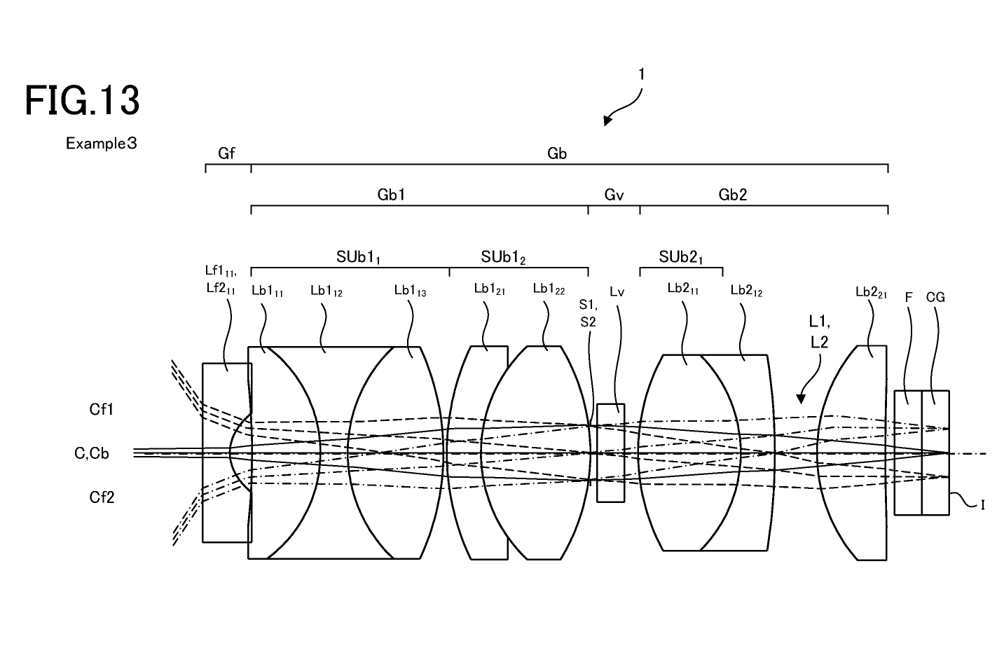

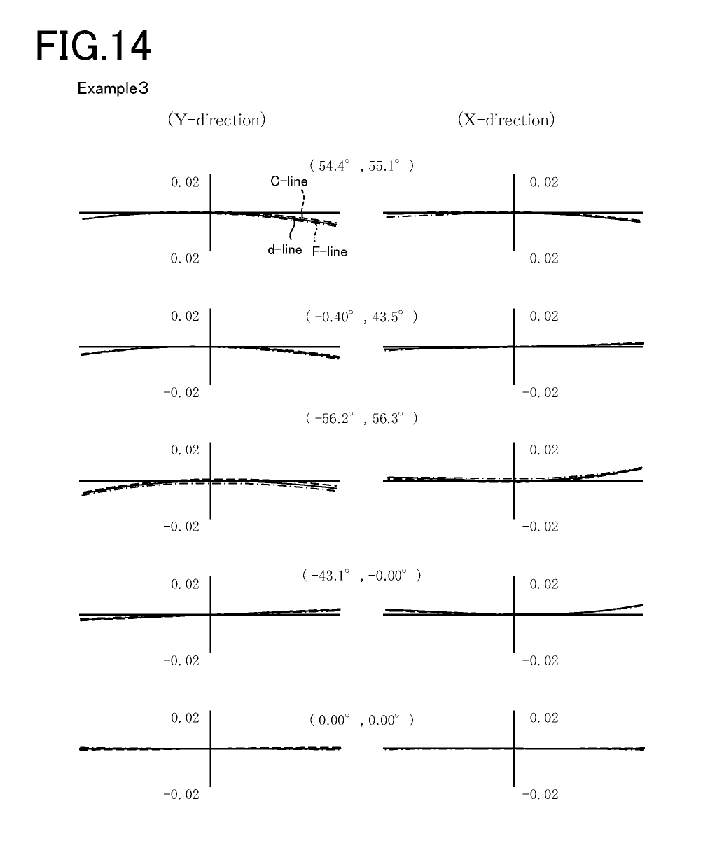

FIG. 12 is a cross-sectional view of the stereoscopic imaging optical system 1 of Example 3 taken along a plane including the first front group central axis Cf1 and second front group central axis Cf2. FIG. 13 is a cross-sectional view of the stereoscopic imaging optical system 1 of Example 3 taken along a plane perpendicular to a plane including the first front group central axis Cf1 and second front group central axis Cf2 and including the back group central axis Cb. FIG. 14 is a lateral aberration diagram of the stereoscopic imaging optical system 1 of Example 3. FIG. 11 is a lateral aberration diagram of the stereoscopic imaging optical system 1 of Example 3.

As illustrated in FIG. 12, the stereoscopic imaging optical system 1 according to Example 3 includes a front group Gf and a back group Gb, in order from an object side to an image side. The front group Gf includes a first front group Gf1 having a first front group central axis Cf1 and a second front group Gf2 having a second front group central axis Cf2 extending parallel to the first front group central axis Cf1. The back group Gb has a single back group central axis Cb.

Parallel arrangement of the first front group Gf1 and second front group Gf2 allows stereoscopic observation.

The first front group Gf1 has a flat-concave negative lens Lf1₁₁ whose flat surface faces the object side. The second front group Gf2 has a flat-concave negative lens Lf2₁₁ whose flat surface faces the object side. The first front group Gf1 and second front group Gf2 are preferably formed integrally into the same shape.

The back group Gb includes a back first group Gb1, a back second group Gb2, a first aperture S1, a second aperture S2, a first deflection group Gv1, and a second deflection group Gv2. The back first group Gb1 has: a cemented lens SUb1₁ composed of a negative meniscus lens Lb1₁₁ whose convex surface faces the image plane I side, a concave-concave negative lens Lb1₁₂, and a convex-convex positive lens Lb1₁₃; and a cemented lens sub1₂ composed of a negative meniscus lens Lb1₂₁ whose convex surface faces the object side and a convex-convex positive lens Lb1₂₂. The back second group Gb2 has: a cemented lens SUb2₁ composed of a convex-convex positive lens Lb2₁₁ and a negative meniscus lens Lb2₁₂ whose convex surface faces the image plane I side; and a positive meniscus lens Lb2₂₁ whose convex surface faces the object side. The first aperture S1 is disposed between the back first group Gb1 and back second group Gb2 and centered about a first aperture center CS1 offset from the back group central axis Cb. The second aperture S2 is centered about a second aperture center CS2 disposed at a position plane-symmetric to the first aperture center CS1 with respect to a plane perpendicular to a plane including the first front group central axis Cf1 and second front group central axis Cf2 and including the back group central axis Cb. The first deflection group Gv1 is disposed between the back first group Gb1 and back second group Gb2. The second deflection group Gv2 is disposed at a position plane-symmetric to the first deflection group Gv1 with respect to a plane perpendicular to a plane including the first front group central axis Cf1 and second front group central axis Cf2 and including the back group central axis Cb.

The first aperture S1 and first deflection group Gv1 are disposed adjacent to each other, and second aperture S2 and second deflection group Gv2 are disposed adjacent to each other. In Example 3, the first aperture S1 is disposed on the object side of the first deflection group Gv1, and the second aperture S2 is disposed on the object side of the second deflection group Gv2.

The first and second deflection groups Gv1 and Gv2 of Example 3 are each constituted of a wedge prism shaped optical element whose thickness in the back group central axis Cb direction gradually increases in a direction separating from the back group central axis Cb. Further, the wedge prism shaped optical elements constituting the first and second deflection groups Gv1 and Gv2 of Example 3 are integrally formed. The integrally formed optical element of Example 3 has an object side surface formed into a plane inclined relative to the back group central axis Cb and an image plane side surface formed into a plane inclined relative to the back group central axis Cb.

Further, a filter F and a cover glass CG are disposed immediately in front of the image plane I.

A first light beam L1 that has entered the first front group Gf1 of the front group Gf from an unillustrated first object plane passes through the flat-concave negative lens Lf1₁₁ to exit from the first front group Gf1 and then enters the back group Gb. The first light beam L1 that has entered the back first group Gb1 of the back group Gb passes through the cemented lens SUb1₁ and cemented lens SUb1₂ to exit from the back first group Gb1 and then passes through the first aperture S1. The first light beam L1 that has passed through the first aperture S1 passes through the first deflection group Gv1 and enters the back second group Gb2. The first light beam L1 that has entered the back second group Gb2 passes through the cemented lens SUb2₁ and positive meniscus lens Lb2₂₁ to exit from the back second group Gb2, passes through the filter F and cover glass CG, and reaches the image plane I.

A second light beam L2 that has entered the second front group Gf2 of the front group Gf from an unillustrated second object plane passes through the flat-concave negative lens Lf2₁₁ to exit from the second front group Gf2 and then enters the back group Gb. The second light beam L2 that has entered the back first group Gb1 of the back group Gb passes through the cemented lens SUb1₁ and cemented lens SUb1₂ to exit from the back first group Gb1 and then passes through the second aperture S2. The second light beam L2 that has passed through the second aperture S2 passes through the second deflection group Gv2 and enters the back second group Gb2. The second light beam L2 that has entered the back second group Gb2 passes through the cemented lens SUb2₁ and positive meniscus lens Lb2₂₁ to exit from the back second group Gb2, passes through the filter F and cover glass CG, and reaches the image plane I.

The following describes configuration parameters in the above Examples 1 to 3.

A coordinate system is defined for each surface. A direction directed from an origin O of the coordinate system on which the surface is defined toward the image plane along the central axis is defined as a Z-axis positive direction. A direction directed from the second front group central axis Cf2 toward the first front group central axis Cf1 on the same surface is defined as an X-axis positive direction. A Y-axis positive direction is defined by a right-hand coordinate system.

In the case where, of the optical surfaces forming the optical system in each Example, a specific surface and the subsequent surface form together a coaxial optical system, surface separations are given to them. In addition, radii of curvatures of surfaces, refractive indices of media and Abbe constants are given as usual.

Given to each eccentric surface are an eccentric amount of the coordinate system on which that surface is defined from the origin O (X, Y and Z in the X-, Y- and Z-axis directions) and the angles (α, β, γ(°)) of tilt of the coordinate system for defining each surface with the X-, Y- and Z-axes of the coordinate system defined on the origin as center. Then, the positive α and β mean counterclockwise rotation with respect to the positive directions of the respective axes, and the positive γ means clockwise rotation with respect to the positive direction of the Z-axis. Referring here to the α, β, γ rotation of the center axis of a certain surface, the coordinate system for defining each surface is first α rotated counterclockwise about the X-axis of the coordinate system defined on the origin of an optical system. Then, the center axis of the rotated surface is β rotated counterclockwise about the Y-axis of a new coordinate system. Finally, the center axis is γ rotated clockwise about the Z-axis of a rotated new coordinate system.

Refractive indices and Abbe constants on d-line (wavelength: 587.56 nm) basis are given, and length is given in mm. The eccentric of each surface is expressed by the eccentric amount from the reference surface as described above. The symbol “∞” affixed to the radius of curvature stands for infinity.

Aspheric data used in the present embodiment include data about aspheric lens surfaces. Aspheric surface shape or configuration may be represented by the following formula (a):

Z=(y²/r)/[1+{1−(1+K)·(y/r)²}^{1/2}]+A4y⁴+A6y⁶+A8y⁸+A10y¹⁰ (a)

where z is indicative of an optical axis where the direction of travel of light is positive, and y is indicative of a direction perpendicular to the optical axis.

In the above formula, r is a paraxial radius of curvature, K is the conic coefficient, and A4, A6 and A8 are the 4th, 6th and 8th order aspheric coefficients, respectively. Note here that the symbol “e” indicates that the subsequent numerical value is a power exponent having 10 as a base. For instance, “1.0e-5” means “1.0×10⁻⁵”.

### Example 1

| Surface | Radius of | Surface | | Refractive | Abbe |
|---|---|---|---|---|---|
| No. | curvature | separation | Eccentricity | index | number |
| Object | ∞ | 5.000 | | | |
| plane | | | | | |
| 1 | ∞ | 0.400 | | 1.8830 | 40.7 |
| 2 | Aspheric | 0.400 | | | |
| | surface | | | | |
| | [1] | | | | |
| 3 | ∞ | 0.000 | Eccentricity | | |
| | (virtual | | (1) | | |
| | surface) | | | | |
| 4 | −12.178 | 0.500 | | 1.8830 | 40.7 |
| 5 | 1.935 | 1.200 | | 1.7847 | 25.7 |
| 6 | −2.622 | 0.050 | | | |
| 7 | 5.891 | 0.700 | | 1.8830 | 40.7 |
| 8 | −5.975 | 0.301 | | | |
| 9 | Stop | 0.050 | Eccentricity | | |
| | | | (2) | | |
| 10 | ∞ | 0.400 | Eccentricity | 1.6477 | 33.8 |
| | | | (2) | | |
| 11 | ∞ | 0.150 | Eccentricity | | |
| | | | (3) | | |
| 12 | 14.172 | 0.400 | | 1.9229 | 18.9 |
| 13 | 1.800 | 1.500 | | 1.6516 | 58.5 |
| 14 | −2.747 | 0.207 | | | |
| 15 | 1.987 | 1.300 | | 1.8830 | 40.7 |
| 16 | −2.004 | 0.400 | | 1.9229 | 18.9 |
| 17 | 57.910 | 0.129 | | | |
| 18 | ∞ | 0.400 | | 1.5163 | 64.1 |
| 19 | ∞ | 0.400 | | 1.5163 | 64.1 |
| 20 | ∞ | 0.000 | | | |
| Image | ∞ | | | | |
| plane | | | | | |

Aspheric surface [1]

| | |
|---|---|
| Radius curvature | 0.536 |
| k | −7.2906e−001 |

Eccentricity [1]

| | | | | | |
|---|---|---|---|---|---|
| X | 0.500 | Y | 0.000 | Z | 0.000 |
| α | 0.000 | β | 0.000 | γ | 0.000 |

Eccentricity [2]

| | | | | | |
|---|---|---|---|---|---|
| X | −0.450 | Y | 0.000 | Z | 0.000 |
| α | 0.000 | β | 0.000 | γ | 0.000 |

Eccentricity [3]

| | | | | | |
|---|---|---|---|---|---|
| X | −0.450 | Y | 0.000 | Z | 0.000 |
| α | 0.000 | β | −19.101 | γ | 0.000 |

Specifications

| | |
|---|---|
| Base length (entrance pupil interval) | 1.0 mm |
| Angle of view (diagonal) | 130° |
| Stop diameter | φ0.55 mm |
| Image size | φ1.41 mm(1.00 × 1.00) |
| Focal distance | 0.472 mm |
| Effective Fno | 3.030 |

### Example 2

| Surface | Radius of | Surface | | Refractive | Abbe |
|---|---|---|---|---|---|
| No. | curvature | separation | Eccentricity | index | number |
| Object | ∞ | 5.000 | | | |
| plane | | | | | |
| 1 | ∞ | 0.500 | | 1.8830 | 40.7 |
| 2 | Aspheric | 0.350 | | | |
| | surface | | | | |
| | [1] | | | | |
| 3 | ∞ | 0.000 | Eccentricity | | |
| | (virtual | | (1) | | |
| | surface) | | | | |
| 4 | −7.102 | 0.400 | | 1.8830 | 40.7 |
| 5 | 2.200 | 1.500 | | 1.7618 | 26.5 |
| 6 | −2.430 | 0.578 | | | |
| 7 | 6.072 | 0.800 | | 1.4875 | 70.2 |
| 8 | −3.094 | 0.050 | | | |
| 9 | ∞ | 0.500 | Eccentricity | 1.8830 | 40.7 |
| | | | (2) | | |
| 10 | ∞ | 0.200 | Eccentricity | | |
| | | | (3) | | |
| 11 | Stop | 0.100 | Eccentricity | | |
| | | | (2) | | |
| 12 | 5.538 | 0.500 | | 1.9229 | 18.9 |
| 13 | 1.900 | 1.400 | | 1.7440 | 44.8 |
| 14 | −5.238 | 0.197 | | | |
| 15 | 2.752 | 1.600 | | 1.7847 | 25.7 |
| 16 | −2.200 | 0.400 | | 1.9229 | 18.9 |
| 17 | −6.900 | 0.125 | | | |
| 18 | ∞ | 0.400 | Eccentricity | 1.5163 | 64.1 |
| | | | (4) | | |
| 19 | ∞ | 0.400 | Eccentricity | 1.5163 | 64.1 |
| | | | (4) | | |
| 20 | ∞ | 0.000 | Eccentricity | | |
| | | | (4) | | |
| Image | ∞ | | Eccentricity | | |
| plane | | | (4) | | |

Aspheric surface [1]

| | |
|---|---|
| Radius curvature | 0.413 |
| k | −9.9488e−001 |

Eccentricity [1]

| | | | | | |
|---|---|---|---|---|---|
| X | 0.500 | Y | 0.000 | Z | 0.000 |
| α | 0.000 | β | 0.000 | γ | 0.000 |

Eccentricity [2]

| | | | | | |
|---|---|---|---|---|---|
| X | −0.550 | Y | 0.000 | Z | 0.000 |
| α | 0.000 | β | 0.000 | γ | 0.000 |

Eccentricity [3]

| | | | | | |
|---|---|---|---|---|---|
| X | −0.550 | Y | 0.000 | Z | 0.000 |
| α | 0.000 | β | −22.841 | γ | 0.000 |

Eccentricity [4]

| | | | | | |
|---|---|---|---|---|---|
| X | −0.400 | Y | 0.000 | Z | 0.000 |
| α | 0.000 | β | 0.000 | γ | 0.000 |

Specifications

| | |
|---|---|
| Base length (entrance pupil interval) | 1.0 mm |
| Angle of view (diagonal) | 130° |
| Stop diameter | φ0.60 mm |
| Image size | φ1.41 mm(1.00 × 1.00) |
| Focal distance | 0.394 mm |
| Effective Fno | 3.339 |

### Example 3

| Surface | Radius of | Surface | | Refractive | Abbe |
|---|---|---|---|---|---|
| No. | curvature | separation | Eccentricity | index | number |
| Object | ∞ | 5.400 | | | |
| plane | | | | | |
| 1 | ∞ | 0.400 | | 1.8830 | 40.7 |
| 2 | Aspheric | 0.325 | | | |
| | surface | | | | |
| | [1] | | | | |
| 3 | ∞ | 0.000 | Eccentricity | | |
| | (virtual | | (1) | | |
| | surface) | | | | |
| 4 | −9.943 | 1.000 | | 1.9229 | 18.9 |
| 5 | −1.908 | 0.400 | | 1.8830 | 40.7 |
| 6 | 2.100 | 1.400 | | 1.5927 | 35.3 |
| 7 | −3.604 | 0.050 | | | |
| 8 | 3.582 | 0.500 | | 1.8830 | 40.7 |
| 9 | 2.109 | 1.600 | | 1.6516 | 58.5 |
| 10 | −2.974 | 0.000 | | | |
| 11 | Stop | 0.100 | Eccentricity | | |
| | | | (2) | | |
| 12 | ∞ | 0.400 | Eccentricity | 1.6516 | 58.5 |
| | | | (3) | | |
| 13 | ∞ | 0.200 | Eccentricity | | |
| | | | (4) | | |
| 14 | 2.947 | 1.500 | | 1.5831 | 59.4 |
| 15 | −2.000 | 0.500 | | 1.9229 | 18.9 |
| 16 | −9.018 | 0.617 | | | |
| 17 | 2.347 | 1.000 | | 1.8830 | 40.7 |
| 18 | 31.144 | 0.133 | | | |
| 19 | ∞ | | Eccentricity | 1.5163 | 64.1 |
| | | | (5) | | |
| 20 | ∞ | | Eccentricity | 1.5163 | 64.1 |
| | | | (5) | | |
| 21 | ∞ | | Eccentricity | | |
| | | | (5) | | |
| Image | ∞ | | Eccentricity | | |
| plane | | | (5) | | |

Aspheric surface [1]

| | |
|---|---|
| Radius curvature | 0.575 |
| k | −6.6917e−001 |

Eccentricity [1]

| | | | | | |
|---|---|---|---|---|---|
| X | 0.550 | Y | 0.000 | Z | 0.000 |
| α | 0.000 | β | 0.000 | y | 0.000 |

Eccentricity [2]

| | | | | | |
|---|---|---|---|---|---|
| X | −0.550 | Y | 0.000 | Z | 0.000 |
| α | 0.000 | β | 0.000 | γ | 0.000 |

Eccentricity [3]

| | | | | | |
|---|---|---|---|---|---|
| X | −0.550 | Y | 0.000 | Z | 0.000 |
| α | 0.000 | β | 11.188 | γ | 0.000 |

Eccentricity [4]

| | | | | | |
|---|---|---|---|---|---|
| X | −0.550 | Y | 0.000 | Z | 0.000 |
| α | 0.000 | β | −16.902 | γ | 0.000 |

Eccentricity [5]

| | | | | | |
|---|---|---|---|---|---|
| X | −0.400 | Y | 0.000 | Z | 0.000 |
| α | 0.000 | β | 0.000 | γ | 0.000 |

Specifications

| | |
|---|---|
| Base length (entrance pupil interval) | 1.1 mm |
| Angle of view (diagonal) | 130° |
| Stop diameter | φ0.80 mm |
| Image size | φ1.41 mm(1.00 × 1.00) |
| Focal distance | 0.468 mm |
| Effective Fno | 3.020 |

Values of elements and Conditional formula (1) for the above Examples 1 to 3 are given below.

| | Example 1 | Example 2 | Example 3 |
|---|---|---|---|
| Element d | 2.13 | 2.41 | 2.45 |
| Element Lb | 8.89 | 10.00 | 10.93 |
| Conditional | 4.17 | 4.15 | 4.46 |
| formula (1) | | | |
| Lb/f | | | |

The following describes application examples of the stereoscopic imaging optical system 1 according to the present embodiment.

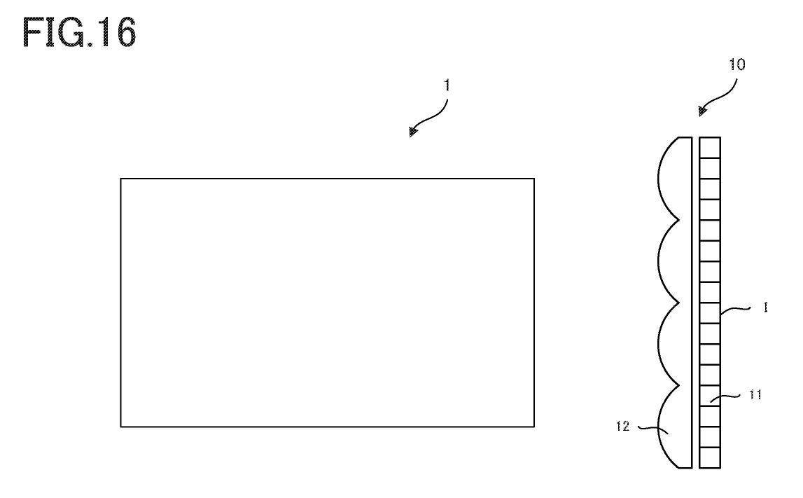

FIG. 16 is a view schematically illustrating an example in which the stereoscopic imaging optical system 1 of the present embodiment is used as a stereoscopic imaging device 10.

The stereoscopic imaging device 10 according to the present embodiment includes a stereoscopic imaging optical system 1 and an imaging device 11. The imaging device 11 is disposed on the image plane I of the stereoscopic imaging optical system 1. A light beam that has passed through the stereoscopic imaging optical system 1 forms an image on the imaging device 11. Thus, stereoscopic imaging can be performed accurately.

The stereoscopic imaging device 10 according to the present embodiment may have a lenticular lens 12 on the object side of the imaging device 11. The presence of the lenticular lens allows more accurate stereoscopic imaging.

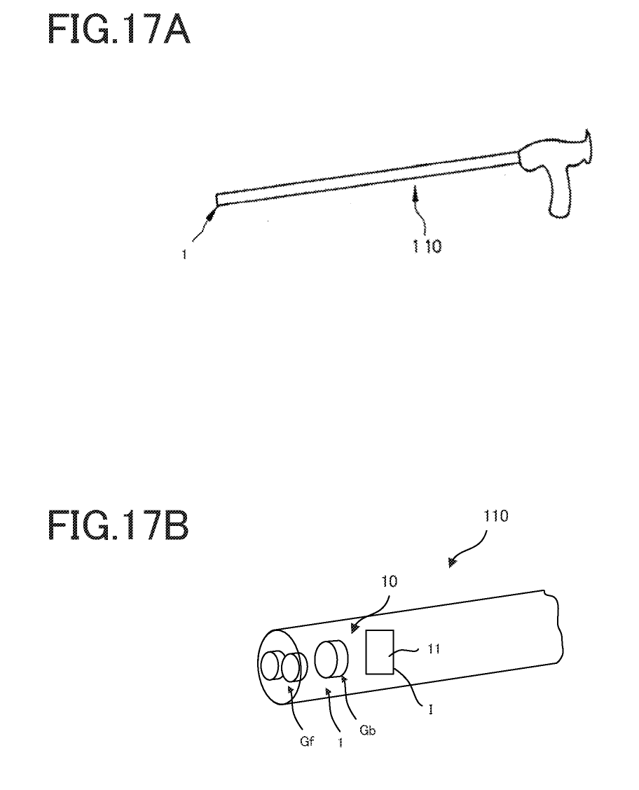

FIGS. 17A and 17B are views illustrating an example in which the stereoscopic imaging optical system 1 according to the present embodiment is used as a stereoscopic imaging optical system 1 attached to a distal end of an endoscope.

FIGS. 17A and 17B are views illustrating an example in which the stereoscopic imaging optical system 1 according to the present embodiment is used as a stereoscopic imaging optical system 1 attached to a distal end of an endoscope 110. FIG. 17A is an example in which the stereoscopic imaging optical system 1 according to the present embodiment is attached to the distal end of a rigid endoscope 110 for stereoscopic imaging/observation of an omnidirectional image. FIG. 17B illustrates a schematic configuration of the distal end of the rigid endoscope 110.

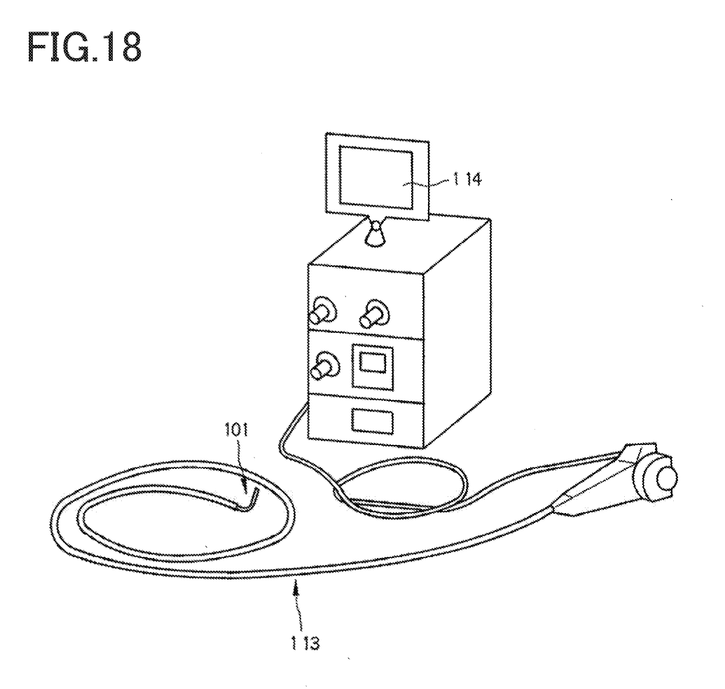

FIG. 18 is an example in which the stereoscopic imaging optical system 1 according to the present embodiment is attached to a distal end of a flexible electronic endoscope 113 so that images taken are stereoscopically displayed on a display device 114 after distortion correction through image processing.

As illustrated in FIG. 18, by using the stereoscopic imaging optical system 1 according to the present embodiment for the endoscope 113, it is possible to stereoscopically image/observe an omnidirectional image and stereoscopically image/observe various regions from angles different from the conventional ones.

While various embodiments of the present invention have been described, it is understood that the invention is not limited only thereto: changes or modifications made to the constructions of such embodiments or some combinations thereof are embraced in the invention as well.

### REFERENCE SIGNS LIST

1: Stereoscopic imaging optical system
Gf: Front group
Gf1: First front group
Cf1: First front group central axis
Gf2: Second front group
Cf2: Second front group central axis
Gb: Back group
Cb: Back group central axis
Gb1: Back first group
Gb2: Back second group
Gv1: First deflection group
Gv2: Second deflection group
S1: First aperture
CS1: First aperture center
S2: Second aperture
CS2: Second aperture center
I: Image plane

1. A stereoscopic imaging optical system comprising in order from an object side to an image plane side:
a front group having a first front group centered about a first front group central axis and a second front group centered about a second front group central axis extending parallel to the first front group central axis; and
a back group centered about a single back group central axis extending parallel to the first front group central axis and second front group central axis,
the back group including:
a back first group on the object side;
a back second group on the image side;
a first aperture disposed between the back first group and back second group and centered about a first aperture center offset from the back group central axis;
a second aperture centered about a second aperture center disposed at a position plane-symmetric to the first aperture center with respect to a plane perpendicular to a plane including the first front group central axis and second front group central axis and including the back group central axis;
a first deflection group disposed between the back first group and back second group; and
a second deflection group disposed at a position plane-symmetric to the first deflection group with respect to a plane perpendicular to a plane including the first front group central axis and second front group central axis and including the back group central axis, wherein:
a first central principal ray of a first light beam that has passed through the first front group passes through the back first group, first aperture center, first deflection group, and the back second group at a position separated from the back group central axis and reaches the image plane, and
a second central principal ray of a second light beam that has passed through the second front group passes through the back first group, second aperture center, second deflection group, and the back second group at a position separated from the back group central axis and reaches the image plane.

2. The stereoscopic imaging optical system according to claim 1, wherein:
the first aperture and first deflection group are adjacent to each other, and
the second aperture and second deflection group are adjacent to each other.

3. The stereoscopic imaging optical system according to claim 1, wherein the first and second deflection groups each include an optical element whose thickness in the back group central axis direction gradually increases in a direction separating from the back group central axis.

4. The stereoscopic imaging optical system according to claim 3, wherein the optical element has a wedge prism shape.

5. The stereoscopic imaging optical system according to claim 1, wherein the first and second front groups include parallel-arranged concave lenses having the same shape.

6. The stereoscopic imaging optical system according to claim 5, wherein the parallel-arranged concave lenses are integrally formed.

7. The stereoscopic imaging optical system according to claim 1, satisfying the following conditional formula (1):
3<fl/d<5 (1)
where fl is an entire length of the optical system, and d is a maximum outer diameter of the optical system.

8. The stereoscopic imaging optical system according to claim 1, wherein an interval between the first front group central axis and second front group central axis is set to equal to or less than 1.2 mm.

9. A stereoscopic imaging device comprising:
the stereoscopic imaging optical system according to claim 1; and
an imaging device.

10. The stereoscopic imaging device according to claim 9, further comprising a lenticular lens on the object side of the imaging device.

11. An endoscope comprising the stereoscopic imaging device according to claim 9.

12. An endoscope comprising the stereoscopic imaging device according to claim 10.

13. A stereoscopic imaging device comprising:
the stereoscopic imaging optical system according to claim 2; and
an imaging device.

14. A stereoscopic imaging device comprising:
the stereoscopic imaging optical system according to claim 3; and
an imaging device.

15. A stereoscopic imaging device comprising:
the stereoscopic imaging optical system according to claim 4; and
an imaging device.

16. A stereoscopic imaging device comprising:
the stereoscopic imaging optical system according to claim 5; and
an imaging device.

17. A stereoscopic imaging device comprising:
the stereoscopic imaging optical system according to claim 6; and
an imaging device.

18. A stereoscopic imaging device comprising:
the stereoscopic imaging optical system according to claim 7; and
an imaging device.

19. A stereoscopic imaging device comprising:
the stereoscopic imaging optical system according to claim 8; and
an imaging device.
